(19) United States
(12) Patent Application Publication
(10) Pub. No.: US 2022/0023471 A1
(43) Pub. Date: Jan. 27, 2022

(54) PROCESS FOR DISINFECTION AND PROTECTING SURFACES FROM CONTAMINATION

(71) Applicant: GERMINATOR MOBILE SANITIZING, LLC, Cumming, GA (US)

(72) Inventors: Jeff Scott Gill, Cumming, GA (US); Frank R. Maresca, Cumming, GA (US)

(73) Assignee: GERMINATOR MOBILE SANITIZING, LLC, Cumming, GA (US)

(21) Appl. No.: 17/377,003

(22) Filed: Jul. 15, 2021

Related U.S. Application Data
(60) Provisional application No. 63/055,701, filed on Jul. 23, 2020.

(51) Int. Cl. A61L 2/18; A61L 2/28; A01N 55/00

(52) U.S. Cl. CPC A61L 2/18; A61L 2/28; A61L 2101/20; A61L 2202/26; A01N 55/00

(57) ABSTRACT

A process for disinfecting a surface comprising at least two steps, step i) comprises applying a first disinfectant to the surface to kill microorganisms and viruses on the surface; and step ii) comprises applying an antimicrobial solution to the disinfected surface to form a prophylactic film to prevent microbial growth. An optional step iii) of testing or monitoring the surface for contamination is also provided.

### BACKGROUND OF THE INVENTION

### Field of the Invention

The present disclosure relates to processes for disinfecting and sterilizing a surface and monitoring to prevent future contamination of the treated surface.

### Description of the Related Technology

Many microorganisms are known to be harmful to humans, as well as other animals that are beneficial to humans, such as pets and farm animals. These microorganisms include, bacteria, viruses, fungi, molds, and mildew, and they can be found on almost all surfaces that humans interact with. Often it is harmful to a person or animal if one of these microorganisms is allowed to enter the human body.

Common disinfectant materials are not one hundred percent effective at killing all the microorganisms on a surface. Any microorganisms that are left alive on a surface can multiply and create a resistant strain of microorganisms that are not killed by the same chemical in the future. Over time, such resistance is more harmful to humans because as greater resistance is achieved, all current methods of preventing the spread of infection on surfaces may become ineffective, at which point humans will be exposed to harmful microorganisms with no means of prevention.

Further, even if a disinfectant is successful at eliminating all of the microorganisms on a particular surface, nothing prevents the attachment of additional microorganisms onto the surface again once the disinfectant has been deactivated or removed from the surface. Therefore, even after a surface has become clean, there is no guarantee that this surface will continue to be free of microorganisms in the future and recontamination or infection may occur at any time after disinfection has been completed.

U.S. Pat. Nos. 9,309,601 and 9,399,823 describe a system for applying a disinfectant that is non-toxic though the use of electrochemical activation of salt-containing water. This system does not address the recontamination of a surface after the initial disinfectant treatment has been applied.

U.S. Pat. No. 10,603,396 describes a process of applying a peracid solution to a surface in two steps to prevent breakdown of the solution overtime in storage. This method is also said to remove safety issues with the formation of peracid solutions through the use of natural disinfectants. Additionally, this two-step process is also said to provide a prophylactic coating in some cases that can protect certain surfaces from corrosion or future microbial contamination. The prophylactic effect is provided by the initial application of the solution to disinfect the surface and the complete removal of contaminants it provides. However, the method of U.S. Pat. No. 10,603,396 does not provide a second solution that can be applied to a clean surface that creates a highly effective barrier to prevent contamination of the surface in the future.

Thus, what is needed is a process for cleaning a surface that can be one hundred percent effective at killing all microorganisms preventing any microbial resistance from occurring. Additionally, the process should also include a surface treatment that is effective in preventing future contamination for a significant period of time. Finally, it would be beneficial if the process contained a step, or steps for monitoring the contamination of the surface, environment, or living being prior to or after it has been initially cleaned so that a person is aware of the failure of the prophylactic treatment after a period of time has passed and that potential contamination of the surface may occur after that time period.

### SUMMARY OF THE INVENTION

In a first embodiment, the disclosure relates to a process for disinfecting a surface comprising two steps. Step a) comprises applying a first disinfectant to the surface to kill microorganisms and viruses on the surface, and Step two comprises applying an antimicrobial solution to the disinfected surface to form a prophylactic film to prevent microbial growth.

In a second embodiment, the disclosure related to the two-step process of disinfecting a surface, and a Step iii) of testing or monitoring the surface for contamination of microbes or live organic matter on the treated surface.

In each of the forgoing embodiments, the first disinfectant may be capable of eliminating germs at a rate of 99.9% or more, and the preferred first disinfectant is HOCl or Hypochlorous acid.

In each of the forgoing embodiments, the first disinfectant may be applied using a battery or electrically powered applicator, which preferably utilizes electrostatic and cold fogging technology.

In each of the forgoing embodiments, the antimicrobial solution of step two may be at least 99% effective at preventing microbial growth on the surface for at least 30 days, more preferably, the antimicrobial solution may be effective at preventing microbial growth on the surface for at least 60 days, and most preferably, the antimicrobial solution may be at least 99% effective at preventing microbial growth on the surface for at least 90 days.

In each of the forgoing embodiments, the antimicrobial solution of step two may comprise an organofunctional trihydroxysilane, which preferably comprises 3-(trihydroxysilyl) propyl dimethyl octadecyl ammonium chloride.

In each of the forgoing embodiments, the second disinfectant may be applied using a battery or electrically powered applicator, which preferably utilizes electrostatic and cold fogging technology.

In each of the foregoing embodiments, the testing or monitoring of step iii) may be conducted beginning 90 days after application of step two.

In each of the forgoing embodiments, the testing or monitoring of step iii) can monitor for the presence of Adenosine triphosphate on the surface.

In each of the forgoing embodiments, the testing or monitoring of step iii) can be provided by an automatic sensor.

In each of the forgoing embodiments, the surface can be at least one of a hard non-porous surface, wood, fabrics, carpeting, draperies, sheets, blankets, bedding, air filters, recirculating air handling systems, apparel, polyurethane foams, roofing materials, shoes, and shoe insoles, shower curtains, throw rugs, toweling, toilet tanks, seat covers, seats, upholstery, vinyl paper, wallpaper, wiping cloths, concrete, and wall board, linens, plastics, stone, porcelain and metals.

### BRIEF DESCRIPTION OF THE DRAWINGS

FIG. 1 is a flow diagram showing the steps of an embodiment of the present invention.

FIG. 2 is a schematic rendering of an automatic sensor that can be used in an embodiment of the present invention.

### DETAILED DESCRIPTION OF THE PREFERRED EMBODIMENT(S)

For illustrative purposes, the principles of the present invention are described by referencing various exemplary embodiments. Although certain embodiments of the invention are specifically described herein, one of ordinary skill in the art will readily recognize that the same principles are equally applicable to, and can be employed in, other systems and methods. Before explaining the disclosed embodiments of the present invention in detail, it is to be understood that the invention is not limited in its application to the details of any particular embodiment shown. Additionally, the terminology used herein is for the purpose of description and not for limitation. Furthermore, although certain methods are described with reference to steps that are presented herein in a certain order, in many instances, these steps can be performed in any order as may be appreciated by one skilled in the art; the novel method is therefore not limited to the particular arrangement of steps disclosed herein.

It must be noted that as used herein and in the appended claims, the singular forms “a”, “an”, and “the” include plural references unless the context clearly dictates otherwise. Furthermore, the terms “a” (or “an”), “one or more”, and “at least one” can be used interchangeably herein. The terms “comprising”, “including”, “having” and “constructed from” can also be used interchangeably.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

It is to be understood that each component, compound, substituent or parameter disclosed herein is to be interpreted as being disclosed for use alone or in combination with one or more of each and every other component, compound, substituent or parameter disclosed herein.

It is also to be understood that each amount/value or range of amounts/values for each component, compound, substituent or parameter disclosed herein is to be interpreted as also being disclosed in combination with each amount/value or range of amounts/values disclosed for any other component(s), compounds(s), substituent(s) or parameter(s) disclosed herein and that any combination of amounts/values or ranges of amounts/values for two or more component(s), compounds(s), substituent(s) or parameters disclosed herein are thus also disclosed in combination with each other for the purposes of this description.

It is further understood that each lower limit of each range disclosed herein is to be interpreted as disclosed in combination with each upper limit of each range disclosed herein for the same component, compounds, substituent or parameter. Thus, a disclosure of two ranges is to be interpreted as a disclosure of four ranges derived by combining each lower limit of each range with each upper limit of each range. A disclosure of three ranges is to be interpreted as a disclosure of nine ranges derived by combining each lower limit of each range with each upper limit of each range, etc. Furthermore, specific amounts/values of a component, compound, substituent or parameter disclosed in the description or an example is to be interpreted as a disclosure of either a lower or an upper limit of a range and thus can be combined with any other lower or upper limit of a range or specific amount/value for the same component, compound, substituent or parameter disclosed elsewhere in the application to form a range for that component, compound, substituent or parameter.

As used herein, the term “surface” refers to both hard and soft surfaces having varying textures and porosities and includes, but are not limited to, glass, metals, sand, granite, wood, fabrics, carpeting, draperies, sheets, blankets, bedding, air filters, air handling systems, apparel, polyurethane foams, roofing materials, shower curtains, rugs, towels, toilet components, upholstery, paper, wallpaper, wiping cloths, tile grout, plaster, drywall, ceramic, cement, clay, bricks, stucco, plastic, tiles, cement, vinyl flooring, and crevices in walls or ceilings. The term surface also includes military equipment, transportation equipment, children's items, plant surfaces, seeds, outdoor surfaces, soft surfaces, air, medical instruments, and the like. The term surface also includes internal and external areas that are part of living beings including by not limited to fur, skin, eggs, egg shells, teeth, gums, and eyes.

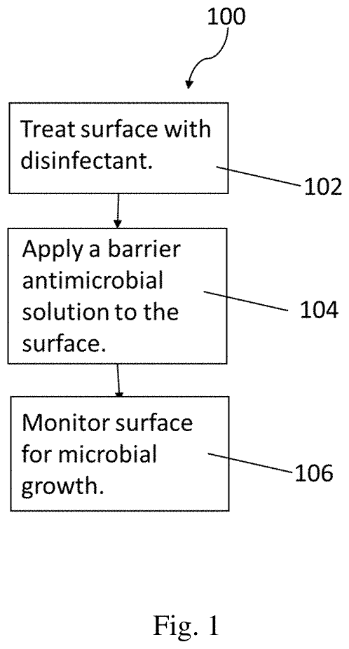

The present invention relates to a process 100 for broad scale disinfection and deodorization of surfaces. See FIG. 1. The process 100 and chemicals used are preferably safe and environmentally friendly. Additionally, the process meets or more preferably exceeds hospital grade results on the most concerning pathogens, including, but limited to MRSA and C-Diff. The process also provides prophylactic persistence in that it is capable of destroying germs long after the application is complete. Preferably, the solutions used in the process do not create bacterial or viral resistance so that the creation of greater issues and a biofilm are avoided. Although the disclosure is directed to application on surfaces, it is also understood that application of Steps one and two may also be to an article of manufacturing that is used to create a surface, including but not limited to within a formula for use as a coating, or on fibers that are used to manufacture a fabric or other material.

In a preferred embodiment, the process 100 comprises at least Step one 102 comprising applying a first disinfectant to the surface to kill microorganisms and viruses on the surface and Step two 104 comprising applying an antimicrobial solution to the disinfected surface formed in Step one 102 to form a prophylactic film to prevent microbial growth. In a more preferred embodiment, a step iii) 106 comprising monitoring the surface for microbes is also utilized. Optional Step iii) 106 comprises monitoring the surface treated in Steps one 102 and two 104 for contamination and failure of the barrier disinfectant. Step one 102 is applied separately from Step two to provide a more effective disinfectant to ensure that all microbes and viruses have been killed prior to the application of the barrier product that may not be as effective at initially killing all organisms when the barrier product is applied by itself. Further, the preferred solutions for use in Step two are not currently registered for virus elimination. Therefore, for the process to be completely effective, Step one 102 uses a chemical that kills viruses.

Step one 102 in the process includes applying a disinfectant utilizing an applicator that is battery or electrically powered. The disinfectant or cleaner has the ability to eliminate germs at a rate of 99.9%, more preferably, the disinfectant has the ability to eliminate germs at a rate of 99.99%, and most preferably the disinfectant has the ability to eliminate germs at a rate of 99.9999% or greater. For example, such disinfectants may include halogen-comprising compounds, quaternary ammonium compounds, metal-comprising, such as gold, copper or silver compounds, sodium peroxide, citric acid, or combinations thereof. It is understood that any disinfectant that can eliminate germs as a rate of 99.9% or the more preferable higher elimination rates can be used for this step of the process. Selection of the specific disinfectant to be used may be dependent on the specific microorganism to be killed, the targeted surface for the application, or potential exposure to humans or animals. In addition to germ elimination, a preferable disinfectant should also be benign to humans and animals.

Preferably, the disinfectant used in step one 102 is HOCl or Hypochlorous acid. HOCl has powerful disinfecting properties and is also benign to humans and pets. Hypochlorous acid is manufactured by an electrolysis process of a salt water solution and is not stable for long-term storage. Due to the inability to store hypochlorous acid, two separate solutions can be mixed prior to use to form the hypochlorous acid. A specific applicator system may be utilized with this disinfectant to mix the two solutions at the time of application. The preferred applicator creates an active solution by mixing salt and water that travels through a brine solution. By creating the final activated product at the site of application, the chemical remains shelf stable for longer periods of time and the active product can be formed directly prior to use.

In addition to the use of a HOCl disinfectant that is created at the time of use through the specific applicator used, other forms of disinfectant may also be used during Step one 102. These other disinfectant products can be in liquid form during storage and can be ready to use straight from storage. Additionally, a solid storage form, such as a tablet, crystal, or powder substance may be used as the disinfectant. Such substances may be added to liquid at the time of application, to create a disinfectant solution that can be applied through the use of an applicator. Again, this process would eliminate any issues that may arise with the storage of the disinfectant chemicals.

The applicator used to apply the disinfectant in Step one 102 may be any applicator that is known in the art and capable of applying a disinfectant in liquid or gaseous form to a surface. However, as noted above with respect to the use of HOCl, depending on the specific disinfectant to be used, a variety of different applicators may be used to apply the disinfectant. Preferably, the applicator is capable of applying the disinfect to a surface that is facing the applicator. More preferably, the applicator applies the disinfectant such that it contacts both the surface facing the applicator, as well as surfaces that are hidden from or not facing the applicator. In a preferred embodiment, the applicator utilizes electrostatic and cold fogging technology. Electrostatic and cold fogging technology allows for accuracy and good coverage of treated areas and allows for material to stick to surfaces using static electricity as well as allows it to reach behind, under and into small spaces without direct contact. Non-electrical and non-battery-operated applicators may also be used to apply the disinfectant. Such applicators include canisters or devices that are known to distribute a chemical throughout an area to apply the chemical to surfaces, such as a pressurized canister.

Step one 102 must be applied prior to the application of Step two 104. The minimum time period between the application of Step one 102 and Step two 104 is sufficient time to allow the solution of Step one 102 to dry. Once the Step one 102 solution is dry, the Step Two 104 solution may be applied. The time required for the solution applied in Step one is dependent on the amount of solution that is applied, the temperature and humidity levels of the location in which it was applied. The time until the solution is dry may range from 30 seconds to a few hours. The solution of Step two 104 should be applied quickly after the solution of Step one 102 has dried so that new microbial growth cannot occur between applications. Preferably, the solution of Step two 104 is applied by a technician immediately upon completion of the application of Step one 102. Preferably, Step two 104 is applied within one minute to 4 hours after the solution of Step one 102 has been applied. More preferably, Step two 104 is applied within 10 minutes to 2 hours after the solution of Step one 102 has been applied.

Step two 104 in the process includes applying an antimicrobial solution to create a microbial barrier on surfaces that will make the areas that were treated uninhabitable for germs to survive. Preferably, the barrier is effective for preventing microbial survival on the treated surface for at least thirty days, and most preferably, the barrier is effective at preventing microbial survival on the treated surface for at least sixty days. More preferable solutions for use in Step two 104 contain no thickeners, stabilizers, colorants, dyes, fragrances or additives. The solution is also preferably non-toxic, non-mutagenic, non-teratogenic and non-allergenic, and safe for use with people, animals and plants. Even more preferably, the solution is non-oxidative and safe for application to a wide variety of surfaces and materials including at least one of a hard non-porous surface, wood, fabrics, carpeting, draperies, sheets, blankets, bedding, air filters, recirculating air handling systems, apparel, polyurethane foams, roofing materials, shoes and shoe insoles, shower curtains, throw rugs, toweling, toilet tanks and seat covers, seats, upholstery, vinyl paper, wallpaper, wiping cloths, concrete, wall board, linens, plastics, stone, porcelain and metals. The solution should also be safe for use on both internal and external areas that are part of living beings including by not limited to fur, skin, eggs, egg shells, teeth, gums, and eyes. Most preferably, the solution used in Step two 104 contains no chlorine and upon microbial interaction will not produce halogenated by-products of disinfection such as carcinogenic trihalomethanes.

The film applied in Step two 104 applies a microbial barrier to affected areas helping control viruses and bacteria and also adds a hydrophobic or water barrier to hard surfaces allowing them to be cleaned quickly and with less effort. The preferred solution of the film covalently bonds (molecular bond) to all surfaces creating a bio-barrier that is uninhabitable for germs and mold to live on.

The antimicrobial solution used in this step can offer the following benefits: application of the solution should provide good durability, broad spectrum, biostatic activity to the surface of a wide variety of substrates. It can also be leach resistant, nonmigrating and not consumed by microorganisms. Further, the antimicrobial solution can be effective against gram positive and gram-negative bacteria, fungi, algae, yeasts and viruses, and can prevent discoloration and deterioration caused by bacteria, fungi, algae and mold. The solution can also inhibit the growth of odor causing bacteria and mildew to prolong the life of the article to which it is applied. Through the inhibited growth, odors can be resisted through a chemical process and not by masking or trapping the odor. The treatment can also be bound to the surface to be protected such that the created film is not destroyed by repeated cleaning or washing.

The surfaces that the antimicrobial solution may be applied to include at least the following surfaces, hard non-porous surfaces such as glass, metals, sand and granite, but also to wood, fabrics, carpeting, draperies, sheets, blankets, bedding, air filters, recirculating air handling systems, outerwear apparel, polyurethane foams (household, industrial and institutional sponges and mops, packaging and cushioning), roofing materials (shingles, roofing granules, wood shakes, felt, stone, synthetic overcoats), shoes and shoe insoles, shower curtains, throw rugs, toweling (cotton, polyester and blends), toilet tank and seat covers, seats, upholstery (acrylics, acetates, canvas, cotton, fiberglass, nylon, polyester, polyethylene, polyolefin, polypropylene, rayon, spandex, vinyl and wool), vinyl paper (for non-food applications), wallpaper, wiping cloths as well as concrete and wall board. The surfaces may also include both internal and external areas that are part of living beings including by not limited to fur, skin, eggs, egg shells, teeth, gums, and eyes.

Preferable antimicrobial solutions useful for this application may contain antimicrobial silanol quaternary ammonium compounds, or antimicrobial essential oils extracted or obtained from the leaves, bark, stems, flowers and berries of plants, and combinations thereof. Some of the preferable essential oils that have demonstrated antimicrobial properties include, but are not limited to: oils of bay, cedar, cinnamon, citronella, clove, eucalyptus, garlic, geranium, lavender, leleshwa, lemon, lemongrass, mint neem, black cumin, onion, oregano, peppermint, rosemary, sandalwood, sesame, tea tree and thyme. A solution containing any combination of one or more of the effective essential oils may be used for Step two 104. Additionally, the antimicrobial solution may contain one or more essential oils in combination with one or more silanol quaternay ammonium compounds. Preferably, the antimicrobial solution contains one or more of the following silanol quaternary ammonium compounds: 3-(trimethoxysilyl) propyl-N-octadecyl-N,N-dimethyl ammonium chloride; 3-(trimethoxysilyl) propyl-N-tetradecyl-N,N-dimethyl ammonium chloride; 3-(trimethoxysilyl) propyl-N-didecyl-N,N-dimethyl ammonium chloride; 3-(triethoxysilyl) propyl-N-octadecyl-N,N-dimethyl ammonium chloride; or 3-(trihydroxysilyl) propyl dimethyl octadecyl ammonium chloride. Most preferably the antimicrobial solution contains 3-(trihydroxysilyl) propyl dimethyl octadecyl ammonium chloride. However, any known antimicrobial solution that is capable of preventing the growth of microbes and/or viruses for at least ninety (90) days can be used for Step two 104. Likewise, the active ingredient weight percent in the mixture can be any amount that is effective in preventing the growth of microbes and/or viruses for at least ninety (90) days. An acceptable range for the active ingredient in the mixture is about 0.1 wt. % to about 10 wt. % and more preferably from about 0.1 wt. % to about 1 wt. %, even more preferably from about 0.75 wt. % to about 0.85 wt. %, with the most preferable being about 0.84 wt. %.

3-(trihydroxysilyl) propyl dimethyl octadecyl ammonium chloride is a water-soluble monomer that on application and drying converts to a bonded polymeric, insoluble surface film. The film binds the active ingredient in the solution to a wide variety of substrates, which increases the spectrum of antiviral, antibacterial and fungal killing power overtime. This solution is U.S. EPA approved for application to many different substrates and articles of varying textures and porosities.

The most preferred antimicrobial solutions, such as those containing 3-(trihydroxysilyl) propyl dimethyl octadecyl ammonium chloride inactivate and kill pathogens through the process of lyses, wherein contact of the pathogens cellular wall with the film applied to the surface produces cellular wall disruption. The contacting cellular wall is broken or ruptured, and the microbe is no longer able to control its internal hydrostatic pressure, resulting in implosion or explosion of the species depending on ambient pressure. No transfer of chemical or chemical residues occurs during this process. As such, the film applied to the surface is not depleted and will continue to lysis cells overtime. There is no increased pathogen resistance to the film on subsequent exposure and no increased mutagenicity.

The antimicrobial barrier solution of Step two 104 may be applied with an applicator that is battery or electrically powered and may be the same or different from the applicator that was used in Step one 102. The applicator should apply the solution used in Step two 104 as a film over the entire treated surface. The applicator used to apply the barrier is any applicator that is capable of applying a liquid to a surface to form a protective layer across the entire surface. Preferably, the applicator utilizes electrostatic and cold fogging technology. Non-electrical and non-battery-operated applicators may also be used to apply the disinfectant. Such applicators include canisters or devices that are known to distribute a chemical throughout an area to apply the chemical to surfaces, such as a pressurized canister.

Step iii) 106 in the process comprises periodic testing or monitoring of surfaces treated with both steps one 102 and two 104 utilizing any device that is capable of testing for live organic matter on the treated surfaces. Step iii) 106 may also include monitoring or testing a surface, the environment, or a living being prior to application of Steps one and two to determine if and what treatment is necessary. Step iii) 106 may utilize either an automatic sensor that continuously monitors for the presence of organic matter on the subject, or a device that requires a test sample be taken from the subject to analyze for the presence of live organic matter. The purpose of periodic testing and monitoring the surface after application of Steps one and two in Step iii) 106 is to verify that the applied surface treatments are maintaining a clean surface and reducing microbial activity on the surfaces. The monitoring also provides an indication of the quality of the cleaning regimen that is being utilized after the surface treatments of steps one 102 and two 104 have been completed.

The periodic monitoring can occur starting 30 days after application of the solution of Step two 104, more preferably, the monitoring begins 60 days after application of the solution of Step two 104, and most preferably the monitoring begins 90 days after the application of the antimicrobial solution in Step two 104. Once monitoring the surface for contamination begins it can be conducted periodically at least every 90 days. More frequent monitoring after the initial 90 days may be preferred to verify that the application of the antimicrobial solution applied in Step two 104 is still effective. Such single-time testing is utilized if a device is used that requires a person to take samples of the treated surfaces. Preferably, a person taking samples for testing would sample one or more random locations on the treated surface, with a concentration of samples being located in any high-touch areas. However, testing of any area of the surface would be beneficial to provide some feedback on how well the surface treatments are working.

Preferable testing systems for use with periodic monitoring are those that measure adenosine triphosphate (ATP) molecules. Luminometers are known for use in measuring ATP and therefore, would be preferable for this testing use. The amount of light generated and sensed by the luminometer is directly proportional to how much ATP is present, which immediately shows the level of biological contamination. Luminometers work with small samples, sometimes with only a few microliters, such as protein solutions or suspension cells contained in a microcentrifuge tube or wells of a microplate and therefore are preferable for this use.

The threshold values for determining when contamination exists depends on the manufacturer, and the specific model of luminometer being used. However, when the threshold value is met, re-treatment of the surface with either or both of steps one 102 and two 104 can be performed to maintain a disinfected surface.

As an alternative and more preferable to the periodic testing described above, a continuous sampling system can be used to detect the presence of living organic matter on the treated surfaces. Preferably, the monitoring system is fully automated and alerts facility personnel by local or remote alarm, telephone, email, or pager when environmental conditions fall outside of established ranges. Alarms can escalate with increasing deviation in the monitored condition and re-alarm at fixed intervals if the condition does not return to its pre-alarm level. Such a continuous, automated sampling system would use an automatic sampler that may be placed in the treated area upon completion of Step two 104, or can be used starting 30, 60 or 90 days after completion of step two 104 to monitor the effectiveness of the antimicrobial surface treatment. The sensor continuously monitors the environment around the treated surface and automatically loads a captured sample into the sensor through the use of non-disposable or multiuse filters or membranes. Such automatic sensors are known to be small in size and can be approximately the size of a quarter, which allows them to be placed in discrete locations that are still in close proximity to higher touched surfaces. In such a scenario, a sampling mechanism is positioned, constantly collecting aerosol and dust particles from either the air, treated surface, or both. The captured sample is automatically sent, at set time intervals, to a biosensor that utilizes a known method to determine if targeted agents were present in the collected sample. Such known methods include but are not limited to optical and electrochemical sensors.

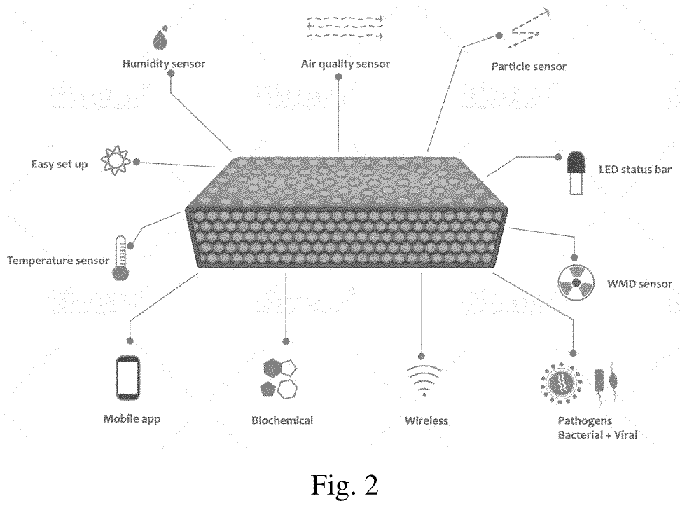

A rendering of a preferred automatic sensor is shown in FIG. 2. One or more of the features shown in the figure can be included in the preferred automatic sensor. The automatic sensor can have a biosensor and/or membrane capable for detecting pathogens, bacterial, viral, or other microorganisms. In addition to the sensor for detecting contamination, one or more of the following may also be present, a humidity sensor, air quality sensor, temperature sensor, radiological sensor, a biochemical sensor, a particle sensor, and other membrane technology. These optional sensors provide feedback to ensure that there is no interference with contamination of the sensor and that the reported results are accurate and may also report the presence of other hazardous conditions.

A wireless transmitter may be used to provide feedback to a remote computer and to possibly create a feedback loop within the entire system. Remote monitoring can be viewed on a dashboard, preferably a dashboard would be part of an application for use on a mobile phone or computer, which would alert the subjects to a contaminated area. The sensor can be easy to setup having simple controls to prepare the communication system, periodicity of testing, and types of alerts to be sent, and location to which the alerts are sent. Additionally, a visual cue, such as a LED status bar, which provides verification that the automatic sensor is still functioning properly can also be present. The visual verification would be used to detect issues with power failure, reporting or sensor failure. If the system is transmitting properly, it may also be setup to report such failures wirelessly to the dashboard.

If the automatic detector senses any contamination of the surface, the dashboard is alerted. At which point, reapplication of either one or both of Steps one 102 and two 104 can be performed to maintain a contamination free surface.

### Examples

Example 1: A Microorganism Efficacy Test for Step One using Hypochlorous acid was tested against a range of different germs and bacteria to determine its efficacy. The hypochlorous acid was added to cultures of the listed microorganisms and left for the indicated amount of time. The percent reduction of each microorganism was measured after the time period had elapsed and the results are shown below in Table 1.

TABLE 1

| MICROORGANISM | TIME POINT | % REDUCTION |
|---|---|---|
| Acinetobacter baumannii | 15 seconds | 100 |
| ATCC 19606 | | |
| Enterococcus faecalis | 15 seconds | 100 |
| (VRE) ATCC 51299 | | |
| Listeria Monocytogens | 60 seconds | >99.96 |
| ATCC 15313 | | |
| Propionibacterium | 60 seconds | >99.99 |
| Acnes ATCC 6919 | | |
| Candida albicans | 60 seconds | >99.9999 |
| ATCC 1023 | | |
| Proteus mirabilis | 15 seconds | >99.999 |
| ATCC7002 | | |
| Serratia marscesens | 15 seconds | >99.999 |
| ATCC 14756 | | |
| Staphylococcus arureus | 15 seconds | >99.999 |
| (MRSA) | | |
| ATCC 33592 | | |
| E. Coli | 60 seconds | >99.999 |
| ATCC 8739 | | |
| Pseudomonas aeruginosa | 60 seconds | >99.999 |
| ATCC 9027 | | |
| S. aureus | 60 seconds | >99.999 |
| ATCC 6538 | | |
| A brasiliensis | 60 seconds | >99.999 |
| ATCC 16404 | | |
| K. pneumonia | 60 seconds | >99.9994 |
| (CRE) ATCC BAA-2146 | | |
| C Difficile | 60 seconds | >99.99990 |
| ATCC 43598 | | |

It is to be understood, however, that even though numerous characteristics and advantages of the present disclosure have been set forth in the foregoing description, together with details of the structure and function of the disclosure, the disclosure is illustrative only, and changes may be made in detail, especially in matters of shape, size and arrangement of parts within the principles of the disclosure to the full extent indicated by the broad general meanings of the terms in which the appended claims are expressed.

What is claimed is:

1. A process for disinfecting a surface comprising steps of:
i) applying a first disinfectant to the surface to kill microorganisms and viruses on the surface; and
ii) applying an antimicrobial solution to the disinfected surface to form a prophylactic film to prevent microbial growth.

2. The process of claim 1 further comprising a step of:
iii) testing or monitoring the surface for microbes.

3. The process of claim 1, wherein the first disinfectant is capable of eliminating germs at a rate of 99.9% or more.

4. The process of claim 4 wherein the first disinfectant is selected from the group consisting of HOCl and hypochlorous acid.

5. The process of claim 1, wherein the first disinfectant is applied using a battery or electrically powered applicator.

6. The process of claim 1, wherein the first disinfectant is applied using an applicator utilizing electrostatic and cold fogging technology.

7. The process of claim 1, wherein the antimicrobial solution of step ii) is at least 99% effective at preventing microbial growth on the surface for at least 30 days.

8. The process of claim 7, wherein the antimicrobial solution is at least 99% effective at preventing microbial growth on the surface for at least 60 days.

9. The process of claim 8, wherein the antimicrobial solution is at least 99% effective at preventing microbial growth on the surface for at least 90 days.

10. The process of claim 1, wherein the antimicrobial solution of step ii) comprises an organofunctional trihydroxysilane.

11. The process of claim 10, wherein the antimicrobial solution comprises 3-(trihydroxysilyl) propyl dimethyl octadecyl ammonium chloride.

12. The process of claim 1, wherein the antimicrobial solution is applied using a battery or electrically powered applicator.

13. The process of claim 1, wherein the antimicrobial solution of step ii) is applied using an applicator utilizing electrostatic and cold fogging technology.

14. The process of claim 2, wherein the testing or monitoring of step iii) is conducted beginning 90 days after application of step ii).

15. The process of claim 2, wherein the testing or monitoring of step iii) monitors for live organic matter on the surface.

16. The process of claim 2, wherein the testing or monitoring of step iii) monitors for the presence of Adenosine triphosphate on the surface.

17. The process of claim 2, wherein the testing or monitoring of step iii) is provided by an automatic sensor.

18. The process of claim 1, wherein the surface is at least one of a hard non-porous surface, wood, fabrics, carpeting, draperies, sheets, blankets, bedding, air filters, recirculating air handling systems, apparel, polyurethane foams, roofing materials, shoes, shoe insoles, shower curtains, throw rugs, toweling, toilet tanks, seat covers, seats upholstery, vinyl paper, wallpaper, wiping cloths, concrete, wall board, linens, plastics, stone, porcelain and metals.
